(19) United States
(12) Patent Application Publication
(10) Pub. No.: US 2014/0187932 A1
(43) Pub. Date: Jul. 3, 2014

(54) CT IMAGING METHOD AND CT SYSTEM BASED ON MULTI-MODE SCOUT SCAN

(71) Applicant: GE MEDICAL SYSTEMS GLOBAL TECHNOLOGY COMPANY, LLC, Waukesha, WI (US)

(72) Inventors: YING LI, Beijing (CN); Yusi CHEN, ChengDu (CN); JING ZHAO, Beijing (CN); DONG WEI, Beijing (CN)

(73) Assignee: GE MEDICAL SYSTEMS GLOBAL TECHNOLOGY COMPANY, LLC, Waukesha, WI (US)

(21) Appl. No.: 14/136,448

(22) Filed: Dec. 20, 2013

(30) Foreign Application Priority Data
Dec. 28, 2012 (CN) 201210581354.0

(51) Int. Cl. A61B 6/00; A61B 6/03

(52) U.S. Cl. CPC A61B 6/482; A61B 6/481; A61B 6/032
USPC 600/431; 378/5

(57) ABSTRACT

A CT imaging method and a CT system based on a multi-mode scout scan. The CT imaging method based on a multi-mode scout scan comprises: performing an instant switching dual energy scout radiation scan on a region of interest of a subject by way of instant switching between high voltage and low voltage to collect dual energy protection data of the region of interest; and reconstructing a material decomposition image and a mono-energetic image based on the collected dual energy projection data.

### TECHNICAL FIELD

Embodiments of the present invention relate to the field of radiograph CT, and more particularly, to a CT imaging method and a CT system based on a multi-mode scout scan.

### BACKGROUND OF THE INVENTION

At present, radiograph CT systems such as X-ray CT system are widely used in various medical institutions for three-dimensional imaging of the regions of interest of the subjects, such as coronary arteries of the subjects, to assist the clinicians to achieve an accurate medical diagnosis of the subjects.

In current coronary artery screening methods, the intracoronary ultrasound area assay is an accurate method to judge the degree of coronary artery stenosis. However, it is not suitable for regular medical application due to high technical condition and cost.

Digital Subtraction Angiograph (DSA) coronary arteriography involves inserting a catheter via thigh femoral artery or other surrounding artery and moving it to ascending aorta before seeking to insert the catheter into a left or right coronary artery opening and injecting contrast media to the coronary artery to develop the coronary artery. The method can clearly show anatomical deformation of coronary artery and position, degree and range of the relevant obstructive pathological changes. Accordingly, DSA coronary arteriography is a method for direct observation of coronary morphology. But it may bring serious side effects to the subjects, such as complications like arrhythmia, embolism and bleeding in sites of puncture. A death rate of the subjects resulting from the complications is about 0.11% to 0.14%, a myocardial infarction rate about 0% to 0.06%, and a myocardial infarction and death rate of the subjects with left coronary main stem stenosis even reach about 3.0%. Moreover, DSA coronary arteriography is an expensive and invasive method that a lot of patients find it hard to accept.

Generally, in CT scan, while all the components of the CT system are maintained stationary, a subject is passed through the CT system to perform a scout scan on the subject to position a region of interest of the subject, thereby identifying the region of interest of the subject for a subsequent complete CT scan. Scout scan is typically performed with low mA; and it provides a projection view along a longitudinal axis of the subject and generally provides aggregations each including internal structure of the subject. However, data collected by the scout scan do not include information sufficient for reconstruction of three-dimensional image, for the projection data in the scout scan are collected along the longitudinal axis of the subject and at a specific angle of projection. In addition, since the scout scan has several overlapping structures in the collected images, it is difficult to identify a specific fine structure of the subject according to the scout scan.

64 slice helical CT, as a noninvasive diagnostic imaging technique, improves the detectable rate of subjects with suspected coronary artery disease; and with high diagnostic accuracy, it can be used as a noninvasive detection method for evaluation and screening of coronary artery stenosis. But 64 slice helical CT requires a higher radiation dose than scout scan. It will become invalid when there is calcification in blood vessels, because calcification in blood vessels will pose serious interference to the injected contrast media.

Gemstone Spectral Imaging (GSI) 64 slice helical CT can solve the calcification problem by separating iodine and calcium via mono-energetic image and material decomposition to remove the interference of blood vessel calcification with diagnosis. However, compared with normal scout scan, all helical CTs including 64 slice helical CT and GSI 64 slice helical CT require a higher radiation dose. Image collected by normal scout scan cannot be used for coronary artery screening due to a low contrast and material overlap.

Therefore, a CT imaging method and a CT system for quickly reconstructing a CT image at a low radiation dose are required.

### SUMMARY OF THE INVENTION

The present invention provides a CT imaging method and a CT system based on a multi-mode scout scan capable of solving the above problems.

According to an embodiment of the present invention, there is provided a radiograph CT imaging system. The radiograph CT imaging system comprises a gantry having an opening; a scan table to support a subject; a radiation source disposed on the gantry and at one side of the subject to emit rays to the subject; a radiation detector disposed on the gantry and at the other side of the subject to detect the rays transmitting through the subject; a radiation controller to control radiation of the radiation source; a data acquisition system disposed on the gantry and coupled to the radiation detector to collect projection data concerning a region of interest of the subject from the rays detected by the radiation detector; an operation console to control operation of one or more of the gantry, the scan table, the radiation controller and the data acquisition system, wherein the operation console is configured to cause the radiograph CT imaging system to perform an instant switching dual energy scout scan on the region of interest of the subject by way of instant switching between high voltage and low voltage, and to reconstruct a material decomposition image and a mono-energetic image corresponding to a predetermined screening purpose from the collected dual energy projection data.

In the radiograph CT imaging system according to an embodiment, the rays are X-rays.

In the radiograph CT imaging system according to an embodiment, the high voltage and the low voltage range between 80 kVp and 140 kVp.

In the radiograph CT imaging system according to an embodiment, the high voltage is 140 kVp and the low voltage is 80 kVp.

In the radiograph CT imaging system according to an embodiment, the high voltage is 120 kVp and the low voltage is 100 kVp.

In the radiograph CT imaging system according to an embodiment, a mono-energetic value corresponding to the reconstructed mono-energetic image ranges between 40 keV and 140 keV.

In the radiograph CT imaging system according to an embodiment, the high voltage and the low voltage switch at a frequency greater than or equal to 500 Hz.

In the radiograph CT imaging system according to an embodiment, the high voltage and the low voltage switch at a frequency of 825 Hz.

In the radiograph CT imaging system according to an embodiment, the operation console is further configured to cause the radiograph CT imaging system to perform a normal scout scan on the subject to position the region of interest of the subject prior to performing the instant switching dual energy scout scan.

In the radiograph CT imaging system according to an embodiment, the predetermined screening purpose is coronary artery stenosis and/or coronary artery calcification.

In the radiograph CT imaging system according to an embodiment, the operation console is further configured to cause the radiograph CT imaging system to perform a scout shuttle scan on the subject in a selected scan range to predict enhanced time of the region of interest subsequent to injecting contrast media to the subject.

In the radiograph CT imaging system according to an embodiment, the operation console is further configured to cause the radiograph CT imaging system to perform an axial or helical shuttle scan on the subject in a selected scan range to predict enhanced time of the region of interest subsequent to injecting contrast media to the subject.

In the radiograph CT imaging system according to an embodiment, based on the predicted enhanced time of the region of interest, the instant switching dual energy scout scan performed by the radiograph CT imaging system on the region of interest of the subject is triggered.

In the radiograph CT imaging system according to an embodiment, materials corresponding to the coronary artery stenosis and the coronary artery calcification are iodine and Hydroxyapatite (HAP).

In the radiograph CT imaging system according to an embodiment, the operation console is further configured to post-reconstruct one or more corresponding material images and mono-energetic images from the collected dual energy projection data based on a screening purpose different from the predetermined screening purpose.

According to an embodiment of the present invention, there is provided a CT imaging method based on a multi-mode scout scan. The method comprises performing an instant switching dual energy scout radiation scan on a region of interest of a subject by way of instant switching between high voltage and low voltage to collect dual energy protection data of the region of interest; and reconstructing a material decomposition image and a mono-energetic image based on the collected dual energy projection data.

In the CT imaging method based on a multi-mode scout scan according to an embodiment, the radiation scan uses X-rays.

In the CT imaging method based on a multi-mode scout scan according to an embodiment, the high voltage and the low voltage range between 80 kVp and 140 kVp.

In the CT imaging method based on a multi-mode scout scan according to an embodiment, the high voltage is 140 kVp and the low voltage is 80 kVp.

In the CT imaging method based on a multi-mode scout scan according to an embodiment, the high voltage is 120 kVp and the low voltage is 100 kVp.

In the CT imaging method based on a multi-mode scout scan according to an embodiment, a mono-energetic value corresponding to the reconstructed mono-energetic image ranges between 40 keV and 140 keV.

In the CT imaging method based on a multi-mode scout scan according to an embodiment, the high voltage and the low voltage switch at a frequency greater than or equal to 500 Hz.

In the CT imaging method based on a multi-mode scout scan according to an embodiment, the high voltage and the low voltage switch at a frequency of 825 Hz.

In the CT imaging method based on a multi-mode scout scan according to an embodiment, the method further comprises selecting a screening protocol for the multi-mode scout scan based on the region of interest of the subject prior to performing the instant switching dual energy scout radiation scan; and performing a normal scout scan on the subject to position the region of interest.

In the CT imaging method based on a multi-mode scout scan according to an embodiment, the method further comprises injecting contrast media to the subject subsequent to positioning the region of interest; and predicting enhanced time of the region of interest.

In the CT imaging method based on a multi-mode scout scan according to an embodiment, the enhanced time of the region of interest is predicted by performing a scout shuttle scan on the region of interest in a selected scan range.

In the CT imaging method based on a multi-mode scout scan according to an embodiment, the enhanced time of the region of interest is predicted by performing an axial or helical shuttle scan on the region of interest in a selected scan range.

In the CT imaging method based on a multi-mode scout scan according to an embodiment, the enhanced time of the region of interest is predicted by using by a user a prediction model based on medical information of the subject.

In the CT imaging method based on a multi-mode scout scan according to an embodiment, based on the predicted enhanced time of the region of interest, the instant switching dual energy scout radiation scan is triggered.

In the CT imaging method based on a multi-mode scout scan according to an embodiment, the reconstructed material decomposition image and mono-energetic image correspond to a predetermined screening purpose.

In the CT imaging method based on a multi-mode scout scan according to an embodiment, the predetermined screening purpose is coronary artery stenosis and/or coronary artery calcification.

In the CT imaging method based on a multi-mode scout scan according to an embodiment, materials corresponding to the coronary artery stenosis and the coronary artery calcification are iodine and HAP.

In the CT imaging method based on a multi-mode scout scan according to an embodiment, the method further comprises: post-reconstructing one or more corresponding material images and mono-energetic images from the collected dual energy projection data based on a screening purpose different from the predetermined screening purpose.

When the CT imaging technique based on a multi-mode scout enhanced scan according to an embodiment of the present invention is adopted, it is unnecessary to perform a complete CT scan on the region of interest of the subject to reconstruct a three-dimensional image of the subject. Therefore, compared with reconstruction of the three-dimensional CT image of the region of interest of the subject, the technique based on a multi-mode scout enhanced scan according to the present invention can greatly reduce an X-ray dose of the subject; moreover, several material images and mono-energetic images corresponding to the screening purpose can be reconstructed from projection data collected by an instant switching dual energy scout scan exposure, such that it is unnecessary to expose the subject many times, thereby further reducing an X-ray dose of the subject and shortening imaging time of the CT image of interest.

### BRIEF DESCRIPTION OF THE DRAWINGS

In the following some exemplary embodiments of the present invention will be described in detail with reference to the accompanying drawings, in which like or similar elements are denoted by the same reference numerals, wherein:

FIGS. 1A-1B show a radiograph CT system according to an exemplary embodiment of the present invention;

FIG. 2 shows a flowchart of a multi-mode scout enhanced scan according to an exemplary embodiment of the present invention performed by the radiograph CT system as shown in FIG. 1;

FIG. 3 shows a flowchart of an instant switching dual energy scout scan according to an exemplary embodiment of the present invention performed by the radiograph CT system as shown in FIG. 1; and

FIG. 4 shows coronary artery CT images obtained from normal scout scan and the multi-mode scout enhanced scan according to the present invention.

### DETAILED DESCRIPTION OF THE INVENTION

In the following detailed description, exemplary embodiments of the present invention are described with reference to the accompanying drawings. However, it will be appreciated by persons skilled in the art that the present invention is not limited to these exemplary embodiments.

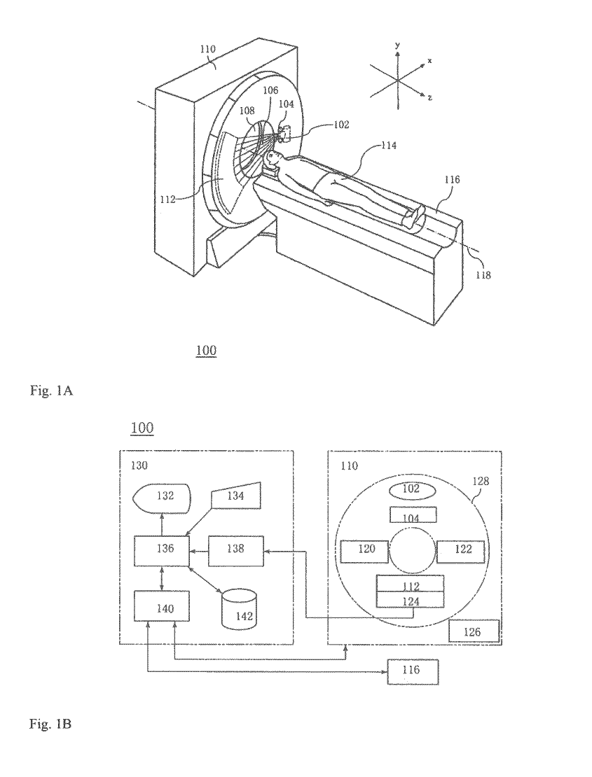

FIGS. 1A-1B show a radiograph CT system 100 according to an exemplary embodiment of the present invention. In one embodiment, the radiograph CT system 100 is an X-ray CT system.

As shown in FIGS. 1A-1B, the X-ray CT system 100 mainly includes three parts: a gantry 110, a scan table 116 for supporting and positioning a subject 114 to be detected, and an operation console 130. The gantry 110 includes an X-ray tube 102. X-rays 106 emitted from the X-ray tube 102 pass through a collimator 104 to form an X-ray beam of such shapes as fan shaped beam and cone shaped beam, to be irradiated to a region of interest of the subject 114. The X-ray beam that passes through the region of interest of the subject 114 is applied to an X-ray detector 112 disposed at the other side of the subject 114. The X-ray detector 112 has a plurality of two-dimensional X-ray detecting elements in the propagation direction (the signal channel direction) and the thickness Z direction (column direction) of the fan-shaped X-ray beam. Optionally, between the X-ray detector 112 and the subject 114 is further provided a collimation component (not shown in FIGS. 1A and 1B), so as to collimate the X-rays passing through the subject 114 before the X-rays impinge against the X-ray detector 112.

A data acquisition system (DAS) 124 is coupled to the X-ray detector 112. The data acquisition system 124 collects the X-rays detected by each of the X-ray detecting elements of the X-ray detector 112 for use as the projection data. The X-ray radiation from the X-ray tube 102 is controlled by an X-ray controller 122. In FIG. 1B, the connections between the X-ray tube 102 and the X-ray controller 122 are not shown.

The data acquisition system 124 collects data related to the tube voltage and tube current applied to the X-ray tube 102 by the X-ray controller 122. In FIG. 1B, the connections between the X-ray controller 122 and the data acquisition system 124 are omitted.

A collimator 104 is controlled by a collimator controller 120. In one embodiment, the collimator 104 and the collimator controller 120 are two separate components. In another embodiment, the collimator controller 120 may be disposed within the collimator 104. In FIG. 1B, the connections between the collimator 104 and the collimator controller 120 are omitted.

Components like the X-ray tube 102, the collimator 104, the X-ray detector 112, the data acquisition system 124, the X-ray controller 122 and the collimator controller 120 are mounted in a rotating portion 128 of the gantry 110. The rotating portion 128 rotates under the control of a rotation controller 126. In FIG. 1B, the connections between the rotating portion 128 and the rotation controller 126 are not shown.

Under the action of a drive system, such as a motor, the scan table 116 can be moved together with the subject 114 carried thereon along a longitudinal axis 118 of the subject into an opening 108 of the gantry 110, so that the region of interest of the subject 114 is substantially perpendicular to the X-ray beam irradiated thereon through the collimator 104.

The operation console 130 has a central processor 136, such as a computer. A control interface 140 is connected to the central processor 136. The gantry 110 and the scan table 116 are connected to the control interface 140. The central processor 136 controls the gantry 110 and the scan table 116 via the control interface 140.

The data acquisition system 124, the X-ray controller 122, the collimator controller 120 and the rotation controller 126 in the gantry 110 are controlled via the control interface 140. In FIG. 1B, the separate connections between the relevant parts and the control interface 140 are not shown.

A data acquisition buffer 138 is connected to the central processor 136. The data acquisition system 124 in the gantry 110 is connected to the data acquisition buffer 138. Projection data collected by the data acquisition system 124 are inputted to the central processor 136 via the data acquisition buffer 138.

The central processor 136 uses the projection data inputted from the data acquisition buffer 138 to perform an image reconstruction. In performing image reconstruction, such methods as the filtered back projection method and three-dimensional image reconstruction method can be used. A storage device 142 is connected to the central processor 136. The storage device 142 may be used to store data, reconstructed images and procedures for implementing the various functions of the X-ray CT system 100.

A display device 132 and an input device 134 are connected to the central processor 136, respectively. The display device 132 displays the reconstructed images and other information output from the central processor 136. A radiologist can input various instructions and parameters to the central processor 136 via the input device 134. Through the display device 132 and the input device 134, the radiologist can achieve an interactive operation of the X-ray CT system 100.

The CT system 100 as shown in FIG. 1 may include CT imaging system(s) of different capabilities, multi-energies and/or dual energies. Correspondingly, these CT imaging systems can be referred to as EDCT, MECT and/or DE-CT imaging systems. In one embodiment, EDCT, MECT and/or DE-CT imaging systems are configurable to adopt different X-ray spectra. For instance, a conventional third-generation CT imaging system can collect projection data of a region of interest at different kVp voltages by turns; and variations involved therein concern energy peaks and spectra of incident photons of the emitted X-ray beams. Since the X-ray detector 112 is sensitive to energy, each photon reaching the X-ray detector 112 is recorded in the form of its photon energy.

Photon energies are detected via scan with two different energy spectra and energy accumulation in the X-ray detector 112, so that projection data of the region of interest can be obtained. EDCT/MECT/DE-CT provides energy differentiation and material characteristics. For instance, in the absence of target scattering, EDCT, MECT and/or DE-CT imaging systems can derive different energy behaviors based on signals from the following two photon energy regions in spectra, i.e., low energy portion and high energy portion of incident X-ray spectra.

The dual energy scan described above is intended to obtain a CT image which can adopt two scans with heterochrosis energy states to promote separation of contrast media in the image. The dual energy scan may include two scans collected in one of the following manners, i.e., the manner of successive time approximation, in which the two scans need to rotate around the subject 114 twice; and the manner of interweaving according to an angle of rotation which is conducted around the subject 114 once, in which the X-ray tube 102 operates at an electric potential, for example, 80 kVp and 140 kVp.

Projection data collected by the dual energy scan can be used to generate a basis material density image and a monochromatic image. The monochromatic image shows the effect of performing CT scan with an ideal monochromatic X-ray tube. When a pair of material density images is given, basis material density image can be generated. For instance, density images of a different material pair, for example, calcium and gadolinium, can be generated according to water and iodine images of a same region of interest. Alternatively, a pair of monochromatic images with respective specific X-ray energies can be generated by using a pair of basis material images. Likewise, a pair of basis material images or a pair of monochromatic images with different energies can be obtained from a pair of monochromatic images.

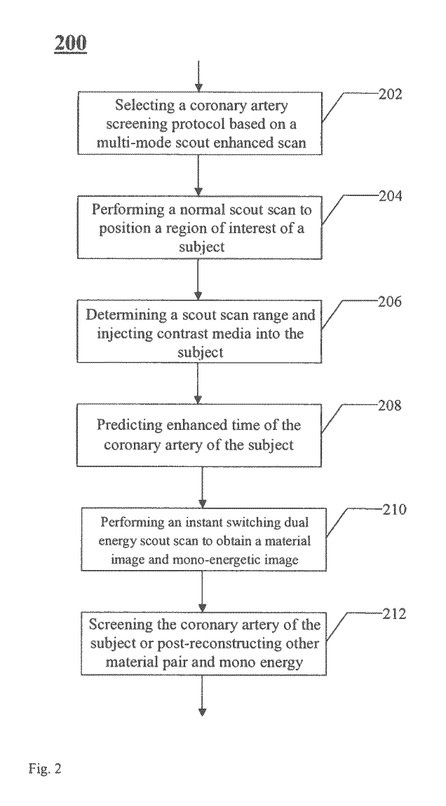

FIG. 2 shows a flowchart of a multi-mode scout enhanced scan according to an exemplary embodiment of the present invention performed by the radiograph CT system 100.

Hereinafter, coronary artery screening is exemplified to describe the CT imaging technique based on a multi-mode scout scan according to the present invention. However, it will be appreciated by persons skilled in the art that the present invention is not limited to the coronary artery screening. For instance, the CT imaging technique based on a multi-mode scout scan according to the present invention can be applied to different screening purposes, such as gallstone and renal calculus.

As shown in FIG. 2, in 202, a coronary artery screening protocol based on a multi-mode scout enhanced scan is selected for the subject 114. In 204, a normal scout scan is performed on the subject 114 so as to position the region of interest including coronary artery of the subject 114. In 206, the scout scan range of the subject 114 is determined, and then contrast media are injected into the body of the subject 114. In 208, after the contrast media are injected into the subject 114, coronary artery enhanced time of the subject 114 is predicted. In 210, in the predicted coronary artery enhanced time of the subject, a fast switching dual energy scout scan is performed on the subject 114 so as to obtain a material image and a mono-energetic image for radiologist or clinician's screening of coronary artery of the subject 114. In 212, the radiologist or clinician carries out a preliminary screening on whether there is stenosis and/or calcification in the coronary artery of the subject 114, or conducts post-reconstruction to select other material pair and mono-energetic energy.

To be specific, in 202, the coronary artery screening protocol based on a multi-mode scout enhanced scan can be selected for the subject 114 via the input device 134. For instance, at least one of coronary artery stenosis and coronary artery calcification is selected according to different screening purposes of the subject 114 given by the clinician. As for a different screening purpose which is not directed to the coronary artery, a different screening protocol can be further selected, and different scout scan and CT image reconstruction parameters can be arranged.

If the screening purpose is coronary artery stenosis and coronary artery calcification, iodine and HAP close to the composition of calcium in human body can be selected as material pair. The iodine-based image matching with HAP can remove the interference from the coronary artery calcification to coronary artery stenosis judgment. The optimal mono energy can be selected based on the principle of maximum CNR.

Additionally, to get the soft tissue and the enhanced blood vessel well separated, water and calcium (or iodine) can be selected as material pair.

Subsequent to selecting the coronary artery screening protocol via the input device 134, the CT imaging system 100 can be initiated to perform a normal-mode scout scan on the subject 114, so as to position the region of interest (for example, chest cavity of the subject 114) including the coronary artery.

To be specific, scan range of the normal scout scan is arranged via the input device 134, such that the CT image reconstructed during the normal-mode scout scan includes enhanced whole coronary artery, thereby meeting the requirement for coronary artery screening. In the process of initiating the CT imaging system 100 to perform a normal-mode scout scan on the subject 114 to position the region of interest of the subject 114, while the gantry 110 is stationary, the scan table 116 carrying the subject 114, driven by a scan table motor, passes through the gantry 110 via the opening 108 at a steady speed. The X-ray tube controller 122 controls the X-ray tube 102 to radiate X-rays to the region of interest of the subject 114. Meanwhile, the data acquisition system 124 obtains projection data by carrying out synchronous sampling of the X-rays detected by the X-ray detector 112, and temporarily stores the obtained projection data within the data acquisition buffer 138. In order to reduce negative impact of X-ray dose on the subject 114, operating current in the X-ray tube 102 can be made by the X-ray tube controller to be at mA order of magnitude. The central processor 136 in the operation console 130 uses the projection data temporarily stored in the data acquisition buffer 138 to generate or reconstruct scout scan image of the subject 114, and based on the generated or reconstructed scout scan image, positions the region of interest of the subject 114 (i.e., chest cavity of the subject) along Z-axis direction and X-axis direction.

Subsequent to determining the position of the region of interest of the subject 114, a range of the region of interest on which a subsequent scout scan is performed is arranged via the input device 134, followed by setting a contrast medium injection protocol according to individual information of the subject 114 and injecting contrast media into the body of the subject 114. Therein, the contrast medium injection protocol can be arranged according to current statistics of enhanced cardiac scan or clinical experience.

After the predetermined time of injecting the contrast media into the subject 114, the coronary artery enhanced time can be predicted.

In an exemplary embodiment, the approximate moment for enhancement of the coronary artery of the subject 114 can be predicted according to medical information of the subject 114, such as height, weight and cardiac output, in combination with the prediction model of coronary artery enhancement.

In another exemplary embodiment, the CT system 100 is initiated to perform a shuttle-mode scout scan on the subject 114 so as to carry out real-time tracking of coronary artery enhancement of the subject 114.

To be specific, scan range of the shuttle-mode scout scan can be arranged via the input device 134. For instance, the scan range can be positioned above the heart of the subject 114 to monitor the aorta enhancement, or the scan range of the region of interest where the heart of the subject 114 is located can be arranged as about 300 mm to get effective enhancement.

The scout shuttle scan is initiated via the input device 134. When the gantry 110 is stationary, the scan table 116 carrying the subject 114, driven by a scan table motor, goes back and forth into the gantry 110 through the opening 108 at a high speed, for example, 150 mm/s. The X-ray tube controller 122 controls the X-ray tube 102 to radiate X-rays to the region of interest of the subject 114. Meanwhile, the data acquisition system 124 obtains projection data by carrying out synchronous sampling of the X-rays detected by the X-ray detector 112, and temporarily stores the obtained projection data within the data acquisition buffer 138.

In the process of performing the scout shuttle scan on the subject 114, by enabling the scan table to move back and forth at a high speed, fast monitoring of coronary artery enhancement can be achieved and X-ray dose radiated on the subject 114 can be reduced. Switching time between different scout scan modes can be reduced by adopting the same scan table movement speed in the scout shuttle scan as in the subsequent instant switching dual energy scout scan. Use of the scout shuttle scan mode can increase frequency of monitoring the coronary artery enhancement. A special small filter (not shown in FIGS. 1A and 1B) directed to cardiac scan can also be used to further reduce X-ray dose radiated on the subject 114.

Coronary artery enhancement based on the scout shuttle scan mode can be automatically triggered on the basis of a predetermined threshold. The predetermined threshold for automatically triggering the coronary artery enhancement can be preset by the radiologist according to clinical experience in combination with the medial registration information of the subject 114. The radiologist can initiate real-time scout scan reconstruction via the input device 134 to trigger enhanced scan. The data acquisition system 124 synchronously collects projection data from the real-time scout scan, and temporarily stores the collected projection data within the data acquisition buffer 138.

The central processor 136 respectively uses the projection data of the first scout shuttle scan temporarily stored within the data acquisition buffer 138 and the projection data of the real-time scout shuttle scan to generate or reconstruct a first scout shuttle scan image and a real-time scout shuttle scan image of the subject 114. Compare the position of coronary artery in the real-time scout shuttle scan image with the corresponding position of coronary artery in the first scout shuttle scan image. If the difference of the two positions exceeds the predetermined threshold, the corresponding position should be recorded and coronary artery enhanced scan should be triggered; moreover, the time for triggering the coronary artery enhanced scan is used as the coronary artery enhanced time. The difference between current position and actual position for coronary artery scan can be used to calculate the delay time of subsequent enhanced scan. The scan switch time of moving components in the CT system 100 can be ignored in predicting the coronary artery enhanced time, because the scout shuttle scan has stationary gantry 110 and uniformed scan table 116 movement speed.

In another exemplary embodiment, normal axial or helical shuttle scan can be initiated via the input device 134 to track coronary enhancement of the subject 114.

To be specific, according to the results of positioning by the normal scout scan the region of interest of the subject 114, section and region of interest for scan tracking can be selected via the input device 134; and real-time axial shuttle scan is used to monitor enhancement of coronary artery in the region of interest. Alternatively, according to the results of positioning by the normal scout scan the region of interest of the subject 114, section and region of interest for scan tracking can be selected via the input device 134; and real-time helical shuttle scan is used to monitor enhancement of coronary artery in the region of interest. The shuttle scan mode can also increase coronary enhancement monitoring frequency.

As for the screening purpose directed to coronary artery, the above disclosure shows a process of injecting contrast media into the subject 114 to perform enhanced scan after positioning the region of interest of the subject 114. However, it shall be understood by persons skilled in the art that different screening purposes involve different needs or doses of contrast media. In other words, as far as the CT imaging technique based on a multi-mode scout scan according to an embodiment of the present invention, it is not essential to inject contrast media to the subject 114 and predict the enhanced time of the region of interest of the subject 114.

At the moment of determining coronary artery enhancement, the CT imaging system 100 is initiated to perform an instant switching dual energy scout scan. If coronary artery enhancement scan is arranged to be automatically triggered based on a predetermined threshold, the instant switching dual energy scout scan mode can be automatically initiated by the central processor 136.

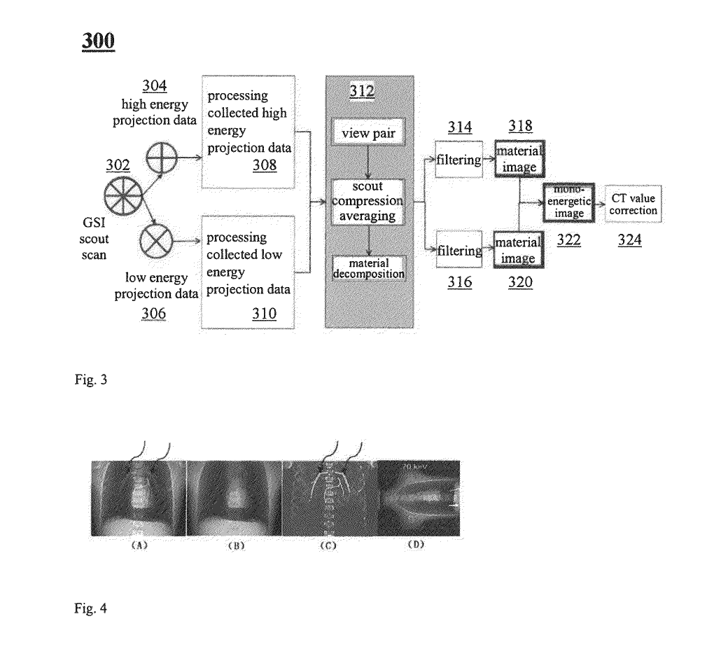

FIG. 3 shows a flowchart of an instant switching dual energy scout scan according to an exemplary embodiment of the present invention performed by the radiograph CT system.

Hereinafter, the instant switching dual energy scout scan performed by the CT system will be detailed with reference to FIG. 3.

When performing the instant switching dual energy scout scan on the subject 114, the CT system 100 carries out scout scan by way of instant switching between high voltage and low voltage, collects dual energy projection data (i.e., high energy projection data and low energy projection data) of the subject 114, generates from the collected dual energy projection data energy spectrum image and material decomposition image of the region of interest of the subject via GSI algorithm, and then generates from the generated energy spectrum image and material decomposition image material pair image and mono-energetic image corresponding to the screening purpose for radiologist or clinician's screening of the subject 114. For instance, if the screening is directed to coronary artery, the generated material pair and mono-energetic image can be used to screen whether the subject 114 suffers from coronary artery stenosis and/or coronary artery calcification.

To be specific, in 302, by enabling the X-ray controller 122 to output a first voltage and a second voltage to the X-ray tube 102 by way of fast switching, the dual energy projection data of the subject 114 are collected, while the gantry 110 is maintained stationary and the subject 114 is passed through the gantry 110 via the opening 108 at a steady speed along the longitudinal axis 118 of the subject 114.

In an exemplary embodiment, the X-ray controller 122 switches the first voltage and the second voltage at a frequency of 825 Hz. In another exemplary embodiment, the X-rat controller 122 switches the first voltage and the second voltage at a frequency equal to or greater than 550 Hz. As the data acquisition system 124 synchronously carries out sampling at a steady scan table 116 speed to fast switch operating voltage of the X-ray tube 102, overlapping projection samples can be obtained based on low kVp configuration and high kVp configuration. In the dual energy scan process, the speed of the scan table 116 can be 100 mm/s, can vary between 100 mm/s and 175 mm/s, or can vary between 0 mm/s and 200 mm/s or more.

The output current of the X-ray tube 102 can be 20 to 400 mA. The second voltage can be greater than the first voltage. Thus, the data acquisition system 124 collects low energy projection data (306) during the first voltage period, and collects high energy projection data (304) during the second voltage period. The first and second voltages can be selected from between 80 kVp and 120 kVp. In an exemplary embodiment, the first voltage can be 80 kVp, and the second voltage can be 140 kVp. In another exemplary embodiment, the first voltage can be 100 kVp, and the second voltage can be 120 kVp. In still another exemplary embodiment, the operating voltage of the X-ray tube 102 can vary continuously during the data acquisition period to generate a plurality of energy levels and equalize the X-ray beams received by the X-ray detector 112.

The data acquisition system 124 transmits the collected dual energy projection data to the data acquisition buffer 138 for temporary storage. The central processor 136 uses the dual energy projection data temporarily stored in the data acquisition buffer 138 to generate or reconstruct one or more dual energy images of the subject 114. The generated or reconstructed dual energy images can be used for generating two-dimensional basis material density image. The basis material density image can be processed to generate specific density image helpful for identifying, characterizing and diagnosing the region of interest in the image. For instance, specific density image can concern bone density, soft tissue, calcium, water, iodine, fat content, and the like.

As shown in FIG. 3, the central processor 136 processes the collected low energy projection data and high energy projection data (308-310). To be specific, the central processor 136 can carry out one or more of the processing manners including format conversion, spits correction, zeros replacement reference, normalization, channel truncation, air calibration, pre bad detector correction, and final bad detector correction on the low energy projection data and high energy projection data extracted from the data acquisition buffer 138.

In 312, the central processor 136 carries out view alignment on the processed low energy and high energy projection data, conducts scout compression, averaging and negative logarithm processing on the aligned high energy and low energy views, and then separates material pair m1 and m2 in the views. In 314 and 316, the central processor 136 filters the separated material pair m1 and m2; in 318 and 320, the CT images of the material pair m1 and m2 are generated or reconstructed according to the views of the filtered material pair m1 and m2; and in 322, mono-energetic images are generated from the CT images of the material pair m1 and m2 based on the selected mono energy. Optionally, the central processor 136 can also correct the generated mono-energetic images based on a predicted CT value.

Prior to or subsequent to collecting projection data of the instant switching dual energy scout scan, material pair and mono energy can be selected for the screening purpose. In an exemplary embodiment, according to the screening purpose, water and calcium (or iodine) can be selected as the material pair m1 and m2. Persons skilled in the art will appreciate that other material pair can be selected, for example, iodine and HAP can be selected as the material pair.

FIG. 4 shows coronary artery CT images obtained from normal scout scan and the multi-mode scout enhanced scan according to the present invention.

In FIG. 4, figure (A) shows a coronary artery CT image obtained from normal scout scan; figure (B) shows a soft tissue CT image concerning coronary artery obtained the multi-mode scout enhanced scan according to the present invention, which CT image corresponds to one of the material images generated by the central processor 136 in 318 and 320 of FIG. 3; figure (C) shows a bone CT image concerning coronary artery obtained from the multi-mode scout enhanced scan according to the present invention, which CT image corresponds to the other one of the material images generated by the central processor 136 in 318 and 320 of FIG. 3; and figure (D) shows a mono energy CT image concerning coronary artery obtained from the multi-mode scout enhanced scan according to the present invention, which CT image corresponds to the mono-energetic image generated from the CT images of the material pair by the central processor 136 in 322 of FIG. 3.

As indicated by arrows in (A) and (C) of FIG. 4, by comparing the scout image obtained from the normal scout scan with the material decomposition image of water and calcium obtained from the GSI scout, the GSI scout scan can more clearly characterize the coronary artery in the obtained CT image via material decomposition, thereby providing visual evidence for diagnosis of coronary artery calcification and/or stenosis.

The optimal keV value which the mono-energetic image as shown in FIG. 4 (D) corresponds to is 70 keV. The optimal keV value can be given to the mono-energetic image according to different rules of screening purpose. The keV value can be self-defined according to medical conditions of the subject and effects of the coronary artery enhanced scan. Lower keV value can be selected, for example, 40 to 60 keV, if higher density resolution is desired. Higher keV value can be selected if beam hardening artifact needs to be removed. More information can be obtained through subtraction or addition processing of the obtained keV values.

The U.S. Pat. No. 8,199,875B2 titled “System and Method of Acquiring Multi-energy CT Imaging Data” and filed by Naveen Chandra, et al. discloses a CT imaging method for using high and low kVp projection data to generate final image. Through citation, the disclosure of said US patent is incorporated in the present disclosure.

According to the coronary artery enhanced CT image generated or reconstructed from the instant switching dual energy GSI scout scan performed by the CT system 100, the radiologist or clinician conducts a preliminary diagnosis on whether the subject 114 suffers from coronary artery stenosis and/or calcification. If intending to simultaneously carry out two and more modes of screening for different screening purposes directed to a same region of interest, the radiologist or clinician can select a different material pair and mono energy to post-reconstruct an image of interest useful for implementation of the corresponding screening from the dual energy projection data collected via the previous instant switching dual energy GSI scout scan. Likewise, if intending to select more materials and mono energies of interest, the radiologist or clinician can also post-reconstruct more images of interest useful for implementation of the corresponding screening from the dual energy projection data collected via the previous instant switching dual energy GSI scout scan.

The selected mono energy which the mono-energetic image as show in FIG. 4 (D) corresponds to is 70 keV. However, the coronary artery of the subject in the mono-energetic image is not clear enough for screening of coronary artery stenosis and/or calcification. Accordingly, lower mono energy can be selected anew to reconstruct a mono-energetic image of higher density resolution from the dual energy projection data collected via the previous instant switching dual energy GSI scout scan, which mono-energetic image of higher density resolution can be used for coronary artery screening.

When the CT imaging technique based on a multi-mode scout enhanced scan according to an embodiment of the present invention is adopted, it is unnecessary to perform a complete CT scan on the region of interest of the subject to generate or reconstruct a three-dimensional image of the subject. Therefore, compared with generation or reconstruction of the three-dimensional CT image of the region of interest of the subject, the multi-mode scout enhanced scan according to the present invention can greatly reduce an X-ray dose radiated on the subject during the CT scan. In addition, by using the CT imaging technique based on a multi-mode scout enhanced scan according to the present invention, several material images and mono-energetic images corresponding to the screening purposes can be generated or reconstructed from projection data collected by an instant switching dual energy scout scan exposure, such that it is unnecessary to expose the subject many times, thereby further reducing an X-ray dose of the subject and shortening imaging time of the CT image of interest.

In an exemplary embodiment according to the present invention, by applying the instant switching dual energy scout scan and the enhanced scan in combination to screening of a subject's coronary artery, a clearer coronary artery image can be quickly obtained at a low X-ray dose, with the purpose of diagnosing coronary artery stenosis and/or calcification.

In an exemplary embodiment according to the present invention, scout shuttle scan is used to track enhancement of the region of interest after contrast media are injected into the subject, thereby guaranteeing accurate monitoring at a low dose. The CT imaging technique based on a multi-mode scout enhanced scan according to the present invention can be applied to any scout scan which needs enhancement.

According to the CT imaging method and CT imaging system of the present invention, a CT image corresponding to a screening purpose can be quickly reconstructed at a low radiation dose.

Although the present invention has been described with reference to specific embodiments, it shall be understood that the present invention is not limited to these specific embodiments. Skilled persons in the art will appreciate that various modifications, substitutions, changes and so on may be made to the present invention. For example, in the above embodiments one step or component may be divided into multiple steps or components; or, on the contrary, a plurality of steps or components in the above embodiments may be realized in one step or one component. All such variations should be within the scope of protection as long as they do not depart from the spirit of the present invention. In addition, the terms as used in the present specification and claims are not limitative, but descriptive. Moreover, according to actual needs, the entire or part of the features described in one specific embodiment can be incorporated into another embodiment.

What is claimed is:

1. A radiograph CT imaging system, comprising:
a gantry comprising an opening;
a scan table to support a subject;
a radiation source disposed on the gantry and at one side of the subject to emit rays to the subject;
a radiation detector disposed on the gantry and at the other side of the subject to detect the rays transmitting through the subject;
a radiation controller to control radiation of the radiation source;
a data acquisition system disposed on the gantry and coupled to the radiation detector to collect projection data concerning a region of interest of the subject from the rays detected by the radiation detector; and
an operation console to control operation of one or more of the gantry, the scan table, the radiation controller, and the data acquisition system,
wherein the operation console is configured to cause the radiograph CT imaging system to perform an instant switching dual energy scout scan on the region of interest of the subject by way of instant switching between a high voltage and a low voltage, and to reconstruct a material decomposition image and a mono-energetic image corresponding to a predetermined screening purpose from the collected dual energy projection data.

2. The radiograph CT imaging system as claimed in claim 1, wherein the rays are X-rays.

3. The radiograph CT imaging system as claimed in claim 1, wherein the high voltage and the low voltage range between 80 kVp and 140 kVp.

4. The radiograph CT imaging system as claimed in claim 1, wherein a mono-energetic value corresponding to the reconstructed mono-energetic image ranges between 40 keV and 140 keV.

5. The radiograph CT imaging system as claimed in claim 1, wherein the high voltage and the low voltage switch at a frequency greater than or equal to 500 Hz.

6. The radiograph CT imaging system as claimed in claim 1, wherein the operation console is further configured to cause the radiograph CT imaging system to perform a normal scout scan on the subject to position the region of interest of the subject prior to performing the instant switching dual energy scout scan.

7. The radiograph CT imaging system as claimed in claim 6, wherein the predetermined screening purpose is coronary artery stenosis and/or coronary artery calcification.

8. The radiograph CT imaging system as claimed in claim 7, wherein the operation console is further configured to cause the radiograph CT imaging system to perform a scout shuttle scan on the subject in a selected scan range to predict enhanced time of the region of interest subsequent to injecting contrast media to the subject.

9. The radiograph CT imaging system as claimed in claim 7, wherein the operation console is further configured to cause the radiograph CT imaging system to perform an axial or helical shuttle scan on the subject in a selected scan range to predict enhanced time of the region of interest subsequent to injecting contrast media to the subject.

10. The radiograph CT imaging system as claimed in claim 8, wherein based on the predicted enhanced time of the region of interest, the instant switching dual energy scout scan performed by the radiograph CT imaging system on the region of interest of the subject is triggered.

11. The radiograph CT imaging system as claimed in claim 7, wherein materials corresponding to the coronary artery stenosis and the coronary artery calcification are iodine and HAP.

12. The radiograph CT imaging system as claimed in claim 1, wherein the operation console is further configured to post-reconstruct one or more corresponding material images and mono-energetic images from the collected dual energy projection data based on a screening purpose different from the predetermined screening purpose.

13. A CT imaging method based on a multi-mode scout scan, the method comprising:
performing an instant switching dual energy scout radiation scan on a region of interest of a subject by way of instant switching between a high voltage and a low voltage to collect dual energy protection data of the region of interest; and
reconstructing a material decomposition image and a mono-energetic image based on the collected dual energy projection data.

14. The method as claimed in claim 13, wherein the radiation scan uses X-rays.

15. The method as claimed in claim 13, wherein the high voltage and the low voltage range between 80 kVp and 140 kVp.

16. The method as claimed in claim 13, wherein a mono-energetic value corresponding to the reconstructed mono-energetic image ranges between 40 keV and 140 keV.

17. The method as claimed in claim 13, wherein the high voltage and the low voltage switch at a frequency greater than or equal to 500 Hz.

18. The method as claimed in claim 13, further comprising:
selecting a screening protocol for the multi-mode scout scan based on the region of interest of the subject prior to performing the instant switching dual energy scout radiation scan; and
performing a normal scout scan on the subject to position the region of interest.

19. The method as claimed in claim 18, further comprising:
injecting contrast media to the subject subsequent to positioning the region of interest; and
predicting enhanced time of the region of interest.

20. The method as claimed in claim 19, wherein the enhanced time of the region of interest is predicted by performing a scout shuttle scan on the region of interest in a selected scan range.
